(19) United States
(12) Patent Application Publication
(10) Pub. No.: US 2025/0169803 A1
(43) Pub. Date: May 29, 2025

(54) BIOPSY CAP FOR AN ENDOSCOPE

(71) Applicant: Boston Scientific Scimed, Inc., Maple Grove, MN (US)

(72) Inventor: Juan M. Alvarez Azpeitia, Framingham, MA (US)

(73) Assignee: Boston Scientific Scimed, Inc., Maple Grove, MN (US)

(21) Appl. No.: 18/960,668

(22) Filed: Nov. 26, 2024

Related U.S. Application Data
(60) Provisional application No. 63/603,402, filed on Nov. 28, 2023.

(51) Int. Cl. A61B 10/04

(52) U.S. Cl. CPC A61B 10/04

(57) ABSTRACT

Devices, systems, and methods for a cap adapted to biopsy ports for an endoscope. Multiple internal features of the rotationally symmetrical bore allow for a tight, operative fit when the cap is placed on any of multiple different biopsy ports, even though the ports have significant differences in both height and shape. Alternating lips and grooves within the contour of the internal bore correspond to different bodies and flanges of the biopsy ports.

### CROSS-REFERENCE TO RELATED APPLICATIONS

This application claims the benefit of U.S. Provisional Patent Application Ser. No. 63/603,402 filed on Nov. 28, 2023, the disclosure of which is incorporated herein by reference.

### FIELD

The present disclosure pertains to endoscopes, endoscope assemblies, guidetubes, introducers, and instrument caps for endoscopes, guidetubes, and introducers. More particularly, the present disclosure pertains to biopsy caps for an access port of an endoscope.

### BACKGROUND

A wide variety of endoscope assemblies and biopsy caps have been developed. Of the known endoscope assemblies and biopsy caps, each has certain advantages and disadvantages. There is an ongoing need to provide alternative endoscope assemblies and biopsy caps as well as methods for making and using the same.

### SUMMARY

This disclosure provides design, material, manufacturing method, and use alternatives for medical devices and medical systems. In a first example, a biopsy cap compatible with a first endoscope biopsy port and a second endoscope biopsy port has a rotationally symmetrical bore having a contoured inner surface. The inner surface defines, in order from the bottom upwards, a first lip, a first groove, a second lip, and a second groove. The first lip has a diameter corresponding to a diameter of a body of the first biopsy port. The first groove has a diameter corresponding to a diameter of an upper flange of the first biopsy port. The second lip has a diameter corresponding to a diameter of an upper body of the second biopsy port. The second groove has a diameter corresponding to a diameter of an upper flange of the second biopsy port. A vertical distance between a bottom surface of the first groove and a top surface of the second groove corresponds to a vertical distance between a bottom surface of the upper flange of the second biopsy port and a top surface of a middle flange of the second biopsy port.

Alternatively or additionally to any of the examples above, the biopsy cap can include a lid having a lower flange and a body, the lower flange having a greater diameter than the body.

Alternatively or additionally to any of the examples above, the contoured inner surface of the rotationally symmetrical bore can further define a top groove above the second groove.

Alternatively or additionally to any of the examples above, the top groove of the contoured inner surface of the rotationally symmetrical bore can have a diameter corresponding to a diameter of the lower flange of the lid.

Alternatively or additionally to any of the examples above, the contoured inner surface of the rotationally symmetrical bore can further define a top lip above the second groove.

Alternatively or additionally to any of the examples above, the top lip of the contoured inner surface of the rotationally symmetrical bore can have a diameter corresponding to a diameter of the body of the lid.

Alternatively or additionally to any of the examples above, the diameter of the first lip can further correspond to a diameter of a lower body of the second biopsy port.

Alternatively or additionally to any of the examples above, the diameter of the first lip can be less than a diameter of a lower flange of the first biopsy port.

Alternatively or additionally to any of the examples above, the diameter of the first lip can be less than a diameter of a lower flange of the second biopsy port.

Alternatively or additionally to any of the examples above, the diameter of the second lip can be less than the diameter of the first lip.

Alternatively or additionally to any of the examples above, the diameter of the second groove can be less than the diameter of the first groove.

In another example, a biopsy cap compatible with a first endoscope biopsy port and a second endoscope biopsy port has a rotationally symmetrical bore having a contoured inner surface. The inner surface defines, in order from the bottom upwards, a first lip, a groove, a second lip, and a second groove. The first lip has a diameter corresponding to a diameter of a body of the first biopsy port. The first groove has a diameter corresponding to a diameter of an upper flange of the first biopsy port. The second lip has a diameter corresponding to a diameter of an upper body of the second biopsy port. The second groove has a diameter corresponding to a diameter of an upper flange of the second biopsy port. The a vertical distance between a bottom surface of the first lip and a top surface of the first groove corresponds to a vertical distance between a bottom surface of a lower flange of the first biopsy port and a top surface of the upper flange of the first biopsy port.

Alternatively or additionally to any of the examples above, the entire biopsy cap can be integrally molded of a single material.

Alternatively or additionally to any of the examples above, the single material can be a polymer or silicone.

As a third example, A method of forming a biopsy cap of any of the preceding examples can include forming the biopsy cap as a solid piece and removing material from the solid piece to form the rotationally symmetrical bore having a contoured inner surface.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 is a perspective view of an illustrative endoscope assembly;

FIG. 2 is an exploded view of a portion of the illustrative endoscope assembly shown in FIG. 1 illustrating a biopsy cap;

FIG. 3 is a perspective view of an illustrative biopsy cap with attached lid;

FIG. 4 is a cross-sectional side view of the biopsy cap;

FIG. 5 is an expanded stratigraphic side view of the interior of the biopsy cap;

FIG. 6 is a cross-sectional perspective view of the biopsy cap fit onto a biopsy port;

FIG. 7 is a cross-sectional side view of the biopsy cap fit onto a biopsy port; and

FIG. 8 is a cross-sectional side view of the biopsy cap fit onto the biopsy port of FIG. 6.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

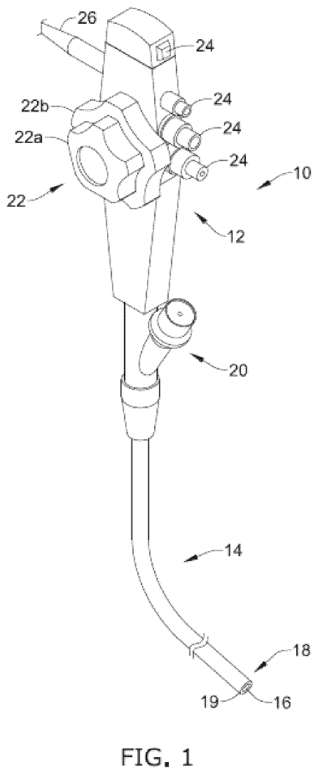

An example endoscope and/or endoscope assembly 10 is illustrated in FIG. 1. The endoscope 10 may be any of a number of types of endoscopes or related medical devices usually identified by the particular anatomy desired to be reached. For example, the endoscope 10 may be a bronchoscope, colonoscope, duodenoscope, esophagoscope, guidetubes, introducers (without or without vision or visualization capabilities), or any other type of endoscope or related medical device. The endoscope 10 may include a handpiece 12 and an elongate shaft 14 extending distally from the handpiece 12 to a distal tip 18. The shaft 14 may include a lumen defining a working channel 16 extending through the shaft 14 from a distal end 19 near the distal tip 18 of shaft 14 to an access port 20 that may be positioned in the handpiece 12 or another portion of the endoscope 10. Although the endoscope 10 is depicted with a single working channel in FIG. 1, it can be appreciated that in other embodiments, the endoscope 10 may include multiple working channels, as desired.

In some cases, the handpiece 12 may include one or a plurality of controls 22, such as rotating knobs, which may be used to control movement of the distal tip 18 of the shaft 14 during operation. For example, a first rotating knob 22a may control up and down movement or deflection of the distal tip 18 of the shaft 14, while a second rotating knob 22b may control side-to-side movement or deflection of the distal tip 18 of the shaft 14. The handpiece 12 may also include one or a plurality of buttons 24, which may be used to activate suction or deliver fluid such as air, saline and/or water, etc. through a lumen of the endoscope 10 or perform other functions as desired. Additionally, in some cases, the handpiece 12 may include an optical cable 26 connected to an external light source (not shown).

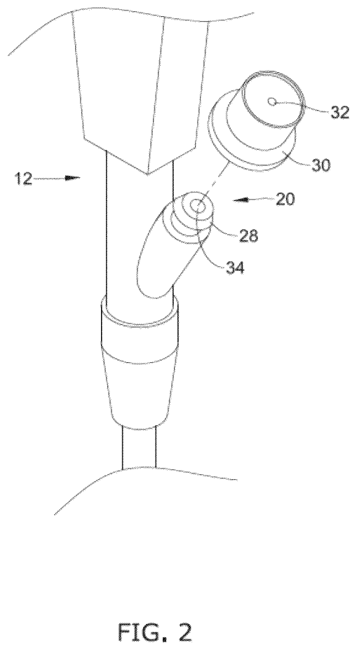

Turning now to FIG. 2, here the access port 20 of the handpiece 12, which provides access to the working channel 16 of the endoscope 10, is illustrated. The access port 20, which may extend from the side of the endoscope 10 or at another location, may include a coupling portion 28 for coupling a cap 30 to the access port 20. The cap 30, which may be removably attached or permanently attached to the access port 20, may provide access for inserting and/or advancing an endoscopic device through working channel 16 of endoscope 10. It will be appreciated that the cap 30 shown in FIG. 1 is intended to be merely illustrative, as the cap 30 may take a variety of different exterior profiles as will be shown in subsequent figures.

In some cases, caps like cap 30, which may be termed “biopsy caps”, may be designed with several functions in mind. For example, the cap 30 may form a fluid/air barrier to the working channel 16 that may help control insufflation and bile fluid egress therefrom that later have the potential to spill onto the clinician's hands and/or the floor thereby interfering with the intervention and/or become a biohazard. In addition, the cap 30 may have an opening 32 extending therethrough. The opening 32 may be in fluid communication with the working channel 16 and it may reduce the size of the opening 34 of working channel 16, for example, to accommodate an endoscopic device or instrument. Thus, caps like cap 30 may be much like an adapter in that it forms a physical transition at opening 34 of working channel 16 (or other instrument channels or access points) so that it transitions to a size more closely to that of the device to be inserted into working channel 16.

A number of additional biopsy caps are contemplated that incorporate at least some of the desirable features of biopsy caps as well as have other desirable characteristics. The forgoing discussion discloses some of the embodiments of caps that are contemplated. These caps may include a passive seal. For the purposes of this disclosure, a passive seal is a seal that seals the endoscope 10 at the port 20 so as to prevent the leakage of bodily fluids and/or air. In addition, by virtue of being “passive”, the caps disclosed herein are configured to seal off the endoscope 10 at the port 20 without the need of any so-called “active” processes or steps by the clinician.

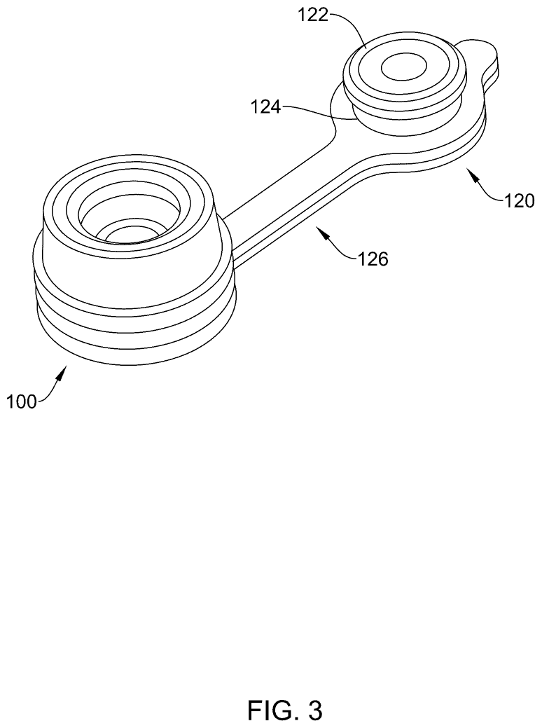

FIG. 3 is a perspective view of a biopsy cap 100 that may, for example, be used in place of the cap 30 shown in FIGS. 1 and 2. The biopsy cap 100 may optionally include an attached lid 120, which if present may include a lower flange 122 and body 124 as shown. The lid 120 is attached to the cap 100 by a strip 126. In some implementations, the lid 120 may be removable from the cap 100, and/or may be otherwise attached.

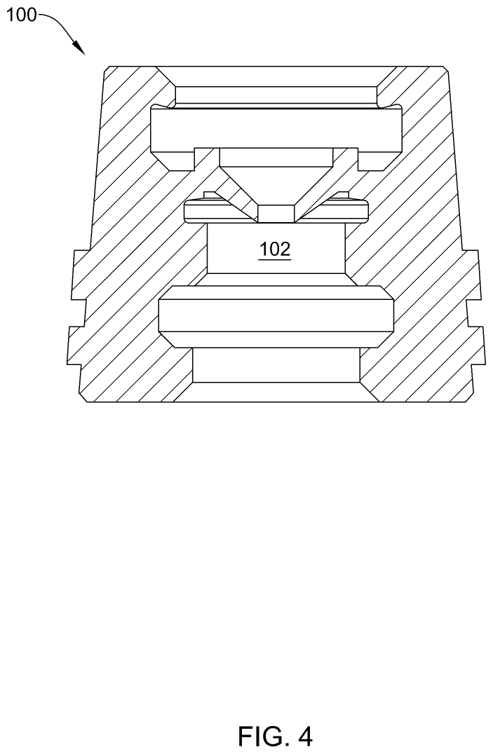

As shown in more detail in FIG. 4, the biopsy cap 100 includes internal features with dimensions that correspond to the dimensions of different endoscopic ports. For purposes of this disclosure, parts are said to have dimensions that “correspond to” one another if one dimension is very slightly larger or smaller than the other to allow the pieces to securely fit together. For example, when an outer piece fits securely around a circular inner piece, the outer piece will have an interior diameter that corresponds to the outer diameter of the inner piece. If two different inner pieces have the same or nearly the same outer diameter, the outer piece may be said to have an inner diameter that corresponds to the outer diameter of either or both inner pieces. Slight variation in the amount of acceptable difference between the dimensions is a known optimization problem that depends on the materials of the pieces as well as their intended use.

The biopsy cap 100 may be of a flexible, resilient material appropriate for creating a tight seal against a biopsy port. The material may be, for example, a polymer such as thermoplastic, or another elastic material such as rubber or silicone. One of ordinary skill can identify the desirable properties of a biopsy cap and select a suitable material, such as a material with a significantly lower durometer and higher resilience than standard rigid materials.

The biopsy cap 100 may be formed or molded of a single piece of material, and then material may be removed to form the internal bore 120. Tooling, cutting, drilling, and/or any other manufacturing process may be used to create the bore 120 from the material. In other implementations, some or all of the internal bore 120 may be included in the die or mold used to initially form the biopsy cap.

As shown in FIG. 4, the biopsy cap 100 includes an internal bore 102 as its main internal feature. The surface of the bore 102 includes several contours to accommodate an endoscopic port at its lower end and the cap lid 120 at its upper end. Both the biopsy cap 100 and its internal bore 102 are rotationally symmetrical about the cap's central vertical axis. This rotational symmetry allows a user to place the biopsy cap 100 over a biopsy port with no need to rotate the cap 100 into a specific orientation.

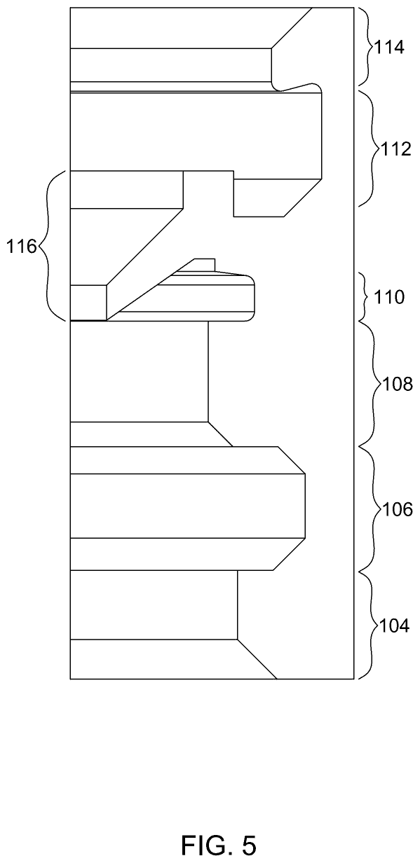

In concordance with its rotational symmetry, the bore 102 can be characterized by lips and grooves representing vertical locations where the bore diameter is lesser and greater, respectively. As shown in FIG. 5, the bore 102 has a first lip 104 at the bottom, followed by a first groove 106, a second lip 108, and a second groove 110. These structures accommodate biopsy ports as further explained below.

An upper lip 114 and upper groove 112 accommodate the lower flange and body of the lid of the biopsy cap. A central channel 116, tapering downward, connects the upper portion of the bore 102 to the lower portion. The channel 116 contains pressure, air, and fluids when using large diameter instruments with the lid 120 open.

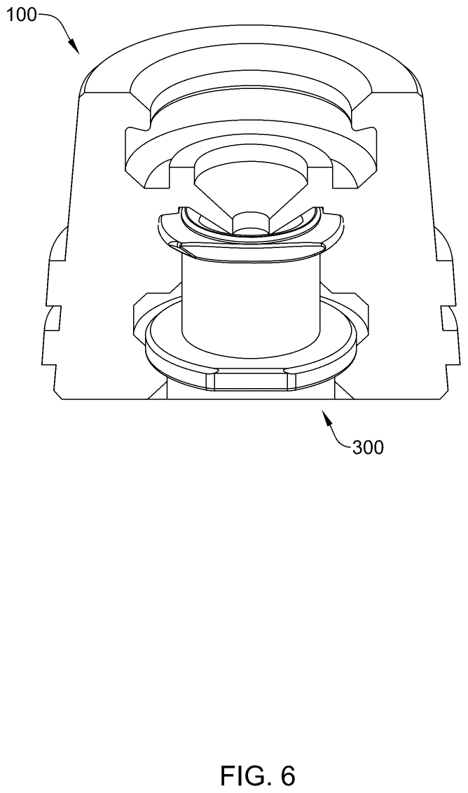

FIG. 6 shows an example of the biopsy cap 100 fitted over a biopsy port 300. As shown, several features of the biopsy cap 100, including the second groove 100 and second lip 108 are sized to match the features of the biopsy port. The fit allows for a secure seal and facilitates the use of the biopsy equipment during an endoscopic procedure.

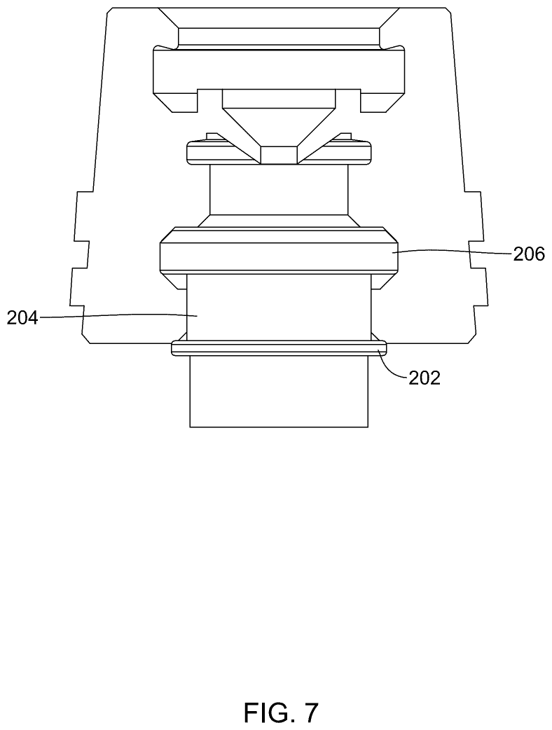

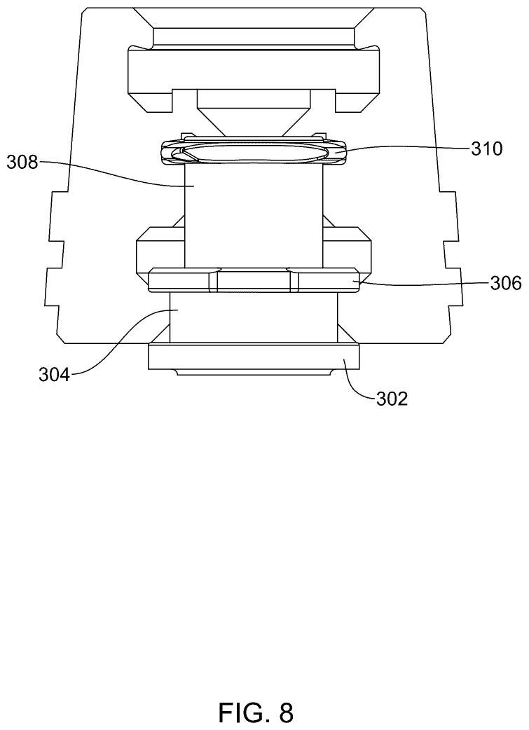

FIGS. 7 and 8 show how the biopsy cap 100 fits both a first biopsy port 200 and the second biopsy port 300. The first biopsy port 200 has a lower flange 202, a body 204, and an upper flange 206. As shown, the top surface of the first groove 106 is shaped to receive the upper flange 206, while the first lip 104 and first groove 106 correspond in height to the height of the port 200 measured from the top surface of the lower flange 202. The diameter of the first groove 106 corresponds to the diameter of the upper flange 206. The diameter of the first lip 104 corresponds to the diameter of the body 204 and is smaller than the lower flange 202 so that the flange 202 abuts the bore opening.

The second biopsy port 300 has a lower flange 302, lower body 304, middle flange 306, upper body 308, and upper flange 310. Although the second biopsy port 300 has more features and is significantly longer than the first biopsy port 200, the lower portion of the second biopsy port 300 fits the same features of the bore 102 as the first biopsy port. The diameter of the first lip 104 corresponds to the diameter of the lower body 304, and the lower surface of the first groove 106 abuts the lower surface of the middle flange 306.

The narrower second lip 108 corresponds in diameter to the upper body 308 of the second biopsy port 300, while the second groove 110 corresponds in diameter to the upper flange 310. The height of the second lip 108 and second groove 110 are selected so that the overall height of the first groove 106, second lip 108, and second groove 110 correspond to the overall height of the middle flange 306, body 308, and upper flange 310. A correct fit is therefore achieved on either the first biopsy port 200 or the second biopsy port 300 with this same cap 100.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A biopsy cap compatible with a first endoscope biopsy port and a second endoscope biopsy port, the cap comprising:
a rotationally symmetrical bore having a contoured inner surface, the inner surface defining, in order from the bottom upwards:
a first lip having a diameter corresponding to a diameter of a body of the first biopsy port,
a first groove having a diameter corresponding to a diameter of an upper flange of the first biopsy port,
a second lip having a diameter corresponding to a diameter of an upper body of the second biopsy port, and
a second groove having a diameter corresponding to a diameter of an upper flange of the second biopsy port;
wherein a vertical distance between a bottom surface of the first groove and a top surface of the second groove corresponds to a vertical distance between a bottom surface of the upper flange of the second biopsy port and a top surface of a middle flange of the second biopsy port.

2. The biopsy cap of claim 1, further comprising a lid having a lower flange and a body, the lower flange having a greater diameter than the body.

3. The biopsy cap of claim 2, wherein the contoured inner surface of the rotationally symmetrical bore further defines a top groove above the second groove.

4. The biopsy cap of claim 3, wherein the top groove of the contoured inner surface of the rotationally symmetrical bore has a diameter corresponding to a diameter of the lower flange of the lid.

5. The biopsy cap of claim 2, wherein the contoured inner surface of the rotationally symmetrical bore further defines a top lip above the second groove.

6. The biopsy cap of claim 5, wherein the top lip of the contoured inner surface of the rotationally symmetrical bore has a diameter corresponding to a diameter of the body of the lid.

7. The biopsy cap of claim 1, wherein the diameter of the first lip further corresponds to a diameter of a lower body of the second biopsy port.

8. The biopsy cap of claim 1, wherein the diameter of the first lip is less than a diameter of a lower flange of the first biopsy port.

9. The biopsy cap of claim 1, wherein the diameter of the first lip is less than a diameter of a lower flange of the second biopsy port.

10. The biopsy cap of claim 1, wherein the diameter of the second lip is less than the diameter of the first lip.

11. The biopsy cap of claim 1, wherein the diameter of the second groove is less than the diameter of the first groove.

12. The biopsy cap of claim 1, wherein the entire biopsy cap is integrally molded of a single material.

13. The biopsy cap of claim 12, wherein the single material is a polymer or silicone.

14. A biopsy cap compatible with a first endoscope biopsy port and a second endoscope biopsy port, the cap comprising:
a rotationally symmetrical bore having a contoured inner surface, the inner surface defining, in order from the bottom upwards:
a first lip having a diameter corresponding to a diameter of a body of the first biopsy port,
a first groove having a diameter corresponding to a diameter of an upper flange of the first biopsy port,
a second lip having a diameter corresponding to a diameter of an upper body of the second biopsy port, and
a second groove having a diameter corresponding to a diameter of an upper flange of the second biopsy port;
wherein a vertical distance between a bottom surface of the first lip and a top surface of the first groove corresponds to a vertical distance between a bottom surface of a lower flange of the first biopsy port and a top surface of the upper flange of the first biopsy port.

15. The biopsy cap of claim 14, further comprising a lid having a lower flange and a body, the lower flange having a greater diameter than the body.

16. The biopsy cap of claim 15, wherein the contoured inner surface of the rotationally symmetrical bore further defines a top groove above the second groove.

17. The biopsy cap of claim 16, wherein the top groove of the contoured inner surface of the rotationally symmetrical bore has a diameter corresponding to a diameter of the lower flange of the lid.

18. A method of forming a biopsy cap, comprising:
forming, as a solid piece, the biopsy cap; and
removing material from the solid piece to form a rotationally symmetrical bore having a contoured inner surface compatible with a first endoscope biopsy port and a second endoscope biopsy port, the inner surface defining, in order from the bottom upwards:
a first lip having a diameter corresponding to a diameter of a body of the first biopsy port,
a first groove having a diameter corresponding to a diameter of an upper flange of the first biopsy port,
a second lip having a diameter corresponding to a diameter of an upper body of the second biopsy port, and
a second groove having a diameter corresponding to a diameter of an upper flange of the second biopsy port,
wherein a vertical distance between a bottom surface of the first groove and a top surface of the second groove corresponds to a vertical distance between a bottom surface of the upper flange of the second biopsy port and a top surface of a middle flange of the second biopsy port.

19. The method of claim 18, wherein the entire biopsy cap is integrally molded of a single material.

20. The method of claim 19, wherein the single material is a polymer or silicone.
